(19) United States
(12) Patent Application Publication
(10) Pub. No.: US 2005/0215865 A1
(43) Pub. Date: Sep. 29, 2005

(54) Method and apparatus for surgical retraction

(75) Inventors: Steven M. LeVahn, Lino Lakes, MN (US); Todd W. Sharratt, Stillwater, MN (US)

Correspondence Address: Z. Peter Sawicki;Westman, Champlin & Kelly, Suite 1600, 900 Second Avenue South, Minneapolis, MN, 55402-3319, US

(73) Assignee: Minnesota Scientific, Inc., St. Paul, MN (US)

(21) Appl. No.: 11/070,836

(22) Filed: Mar. 2, 2005

Related U.S. Application Data
(63) Continuation-in-part of application No. 10/623,179, filed on Jul. 18, 2003.
(60) Provisional application No. 60/396,850, filed on Jul. 18, 2002.

(51) Int. Cl. A61B 1/32

(52) U.S. Cl. 600/231

(57) ABSTRACT

A method of performing surgical retraction of flesh includes positioning a patient on a surgical table and making an incision into the flesh of the patient. A retractor support is mounted to the surgical table and positioned proximate the incision with a blade positioned within the incision and having a flexible member attached to the blade. The retractor blade is positioned manually to retract flesh. The flexible member in tension is attached to the retractor support thereby retracting the flesh and exposing the surgical site.

### CROSS REFERENCE TO RELATED APPLICATIONS

This application is a continuation-in-part of application Ser. No. 10/623,179; filed Jul. 18, 2003, which is hereby incorporated by reference in its entirety, which claims priority of U.S. Provisional Application No. 60/396,850, filed Jul. 18, 2002, which is hereby incorporated by reference in its entirety.

### BACKGROUND OF THE INVENTION

The present invention relates to a method and apparatus for performing surgical retraction of skin and flesh. More particularly, the present invention relates to a method and apparatus for performing surgical retraction with a retractor having a flexible member attached to a table mounted support apparatus.

While the method and apparatus for surgical retraction is useful on any area of the body, one area of the body that the method and apparatus of the present invention is particularly useful is in knee-joint surgery and particularly in knee-joint replacement surgery. The use of rigid retractors during the knee replacement surgery, or any other surgical procedure requiring repositioning of the body, can add time to the surgery. The rigid retractors cannot compensate or adjust along with the movement of the body, therefore the surgeon must unclamp the retractor, reposition the retractor within a surgical site and resecure the retractor to the support apparatus. Depending upon the number of times the body is repositioned during the surgical procedure, the surgeon may become frustrated by the time and effort required to manipulate the surgical clamps and repositioning the surgical retractors may become burdensome to the surgeon.

### SUMMARY OF THE INVENTION

The present invention includes a method of performing surgical retraction of flesh. With the patient lying on a surgical table, an incision is made into the flesh. A retractor support is mounted onto the surgical table. The flesh layers are retracted from the incision with a retractor blade positioned within the incision and having a flexible member attached to the blade. The retractor blade is manually positioned to retract the flesh. The flexible member in tension is secured to the retractor support thereby retaining the flesh and exposing the surgical site.

### BRIEF DESCRIPTION OF THE DRAWINGS

FIG. 1 is a perspective view of an apparatus of the present invention retracting skin and flesh from a knee-joint.

FIG. 2 is a perspective view of the apparatus of the present invention for retracting skin and flesh.

FIG. 3 is a perspective view of a retractor blade of the apparatus of the present invention.

FIG. 4 is a perspective view of a portion of the retractor blade of the apparatus of the present invention attached to a flexible strap.

FIG. 5 is a perspective view of a portion of the flexible strap of the apparatus of the present invention attached to a retractor support arm.

FIG. 6 is a side view of a support arm of the apparatus of the present invention.

FIG. 7 is a perspective view of an alternative embodiment of the apparatus for retracting skin and flesh of the present invention.

FIG. 8 is a perspective view of a portion of the embodiment of FIG. 7.

FIG. 9 is a top view of the embodiment of FIG. 7.

FIG. 10 is a perspective view of a portion of the support arm of the apparatus of the present invention illustrating differently configured notches for use in the present invention.

FIG. 11 is a perspective view of another alternative embodiment of the apparatus of the present invention for retracting skin and flesh.

FIG. 12 is an enlarged perspective view of a portion of the embodiment of FIG. 11.

FIG. 13 is an enlarged perspective view of a portion of the embodiment of FIG. 11.

FIG. 14 is a perspective view of another alternative embodiment of the apparatus of the present invention for retracting skin and flesh.

### DETAILED DESCRIPTION OF THE PREFERRED EMBODIMENTS

The present invention includes a method and an apparatus for performing surgical retraction to expose a surgical site in a patient's body. Throughout this application reference will be made to performing surgical retraction to expose a knee-joint for a knee-joint replacement surgery. However, while the method and apparatus of the present invention are useful in retracting skin and flesh during the knee-joint replacement surgery, the method and apparatus of the present invention are useful during any surgical procedure where skin and flesh are retracted.

The method and apparatus of the present invention utilizes surgical retractors that are secured to a retractor support apparatus that is mounted to a surgical table. Preferably, the method and apparatus for performing surgical retraction allows for movement of the surgical retractor within the surgical site while not requiring the repositioning of the surgical retractors within an incision or re-securing the surgical retractors to the support apparatus during the surgical procedure.

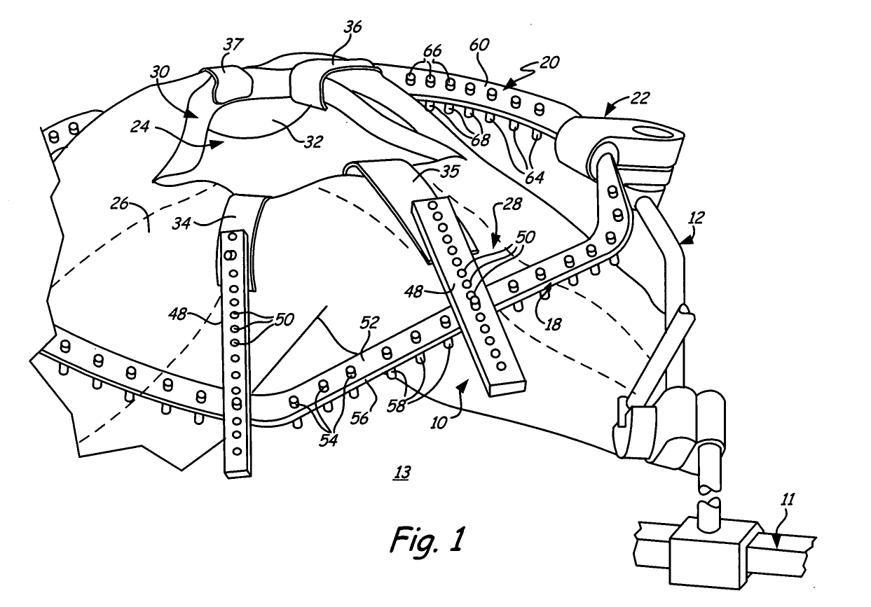

The apparatus of the present invention is generally indicated at 10 in FIG. 1. The apparatus 10 includes a retractor support apparatus 12 that is rigidly mounted to a rail 11 of a surgical table 13 in a manner that is well known in the art and is described in U.S. Pat. Nos. 4,617,916, 4,718,151, 4,949,707, 5,400,772, 5,741,210, 6,042,541, 6,264,396 and 6,315,718 all of which are herein incorporated by reference. From the mount to the surgical table 13, the retractor support apparatus 12 includes left and right second support arms 18 and 20 that extend over the surgical table. The support arms 18 and 20 are independently adjustable into an infinite number of selected positions through use of a clamping mechanism 22 which is described in U.S. Pat. Nos. 5,899,627 and 6,264,396, which are herein incorporated by reference. The support arms 18 and 20 extend in a generally lateral or horizontal direction on opposite sides of a knee-joint 24. The clamp 22 secures the adjustable support arms 18 and 20 in selected angular positions with respect to the knee-joint 24.

The knee-joint 24 is preferably placed in and supported in a bent position as is typically done in knee-joint replacement surgery. The bent position is approximately a 90° angle between a femur 26 and a tibia 28. The support arms 18 and 20 are disposed on opposite sides and below the knee-joint 24.

An incision 30 is made on top of the knee to gain access to the knee-joint 24. The incision is made directly over the patella 32 or on occasion to the left or right of the patella 32 depending on the surgeon's preference, and/or the type of surgical procedure to be performed. Once the incision 30 is made, a plurality of retractors 34, 35, 36 and 37 are positioned in selected positions to manually retract flesh layers to expose the knee-joint 24. By flesh is meant skin, both epidermal and dermal layers, and any underlying subcutaneous tissue, organs and bone. The terms skin and flesh are used herein interchangeably and are intended to have the same meaning. Once in the selected positions, the retractors 34, 35, 36 and 37 are supported by and retained to the adjustable support arms 18 and 20 that are components of the retractor support apparatus 12 that is mounted to the rail 11 of the surgical table 13.

One skilled in the art will recognize that any knee-joint replacement surgical procedure, including a total knee-joint replacement and/or a partial knee-joint replacement, can be performed using the retractors 34, 35, 36 and 37 that are supported and retained by the table mounted retractor support apparatus 12. By total knee-joint replacement is meant a surgical procedure where all of the contacting surfaces of lower end of the femur and the upper end of the tibia and optionally, the patella are replaced. By partial knee-joint replacement is meant a surgical procedure where the worn or damaged contacting surfaces of the lower end of the femur and the upper end of the tibia are replaced while the healthy portion of the knee-joint remains intact. The retractors 34, 35, 36 and 37 are also useful in retracting flesh in other surgical procedures.

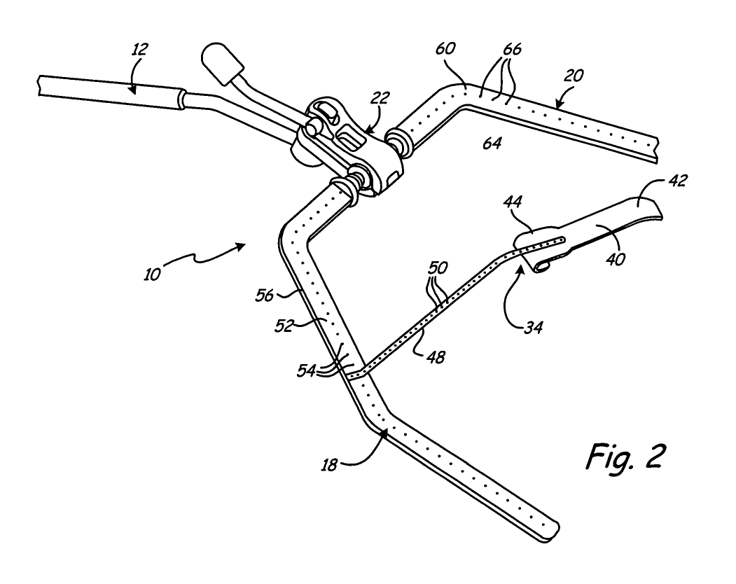

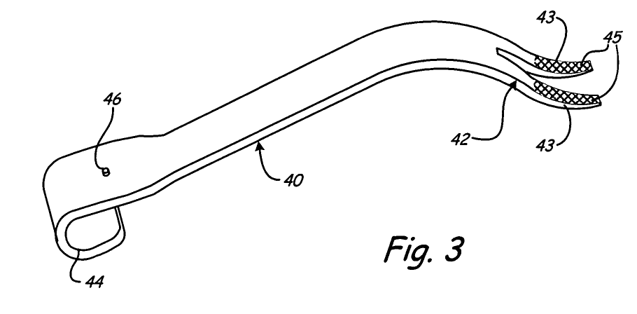

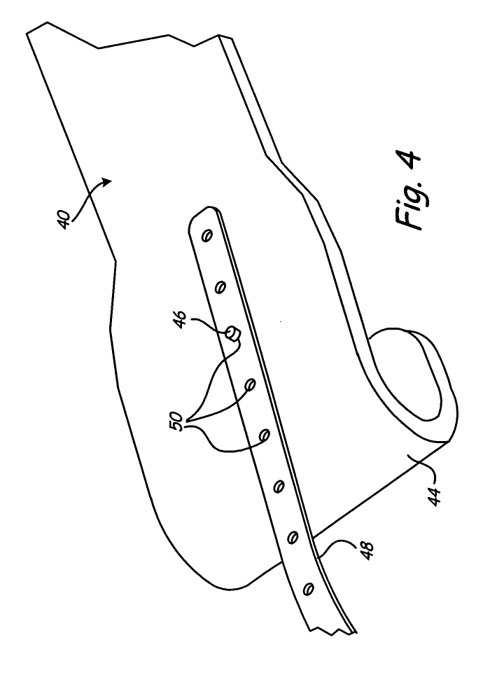

Since surgical retractors 34, 35, 36 and 37 are of the same construction only retractor 34 will be described in detail. Referring to FIGS. 2-4, the retractor 34 includes a retractor blade 40 having a flesh engaging end 42 that is positioned into the incision 30 at the surgical site. The end 42 has a pair of tines 43 that have a knurled or roughened surface 45. A peg 46 proximate a flexible member engaging end 44 of the retractor blade 40 engages one of a plurality of apertures 50 in a flexible strap 48. Manual force is applied to the flexible strap 48 and to the retractor blade 40 to position the peg 46 within one of the plurality of apertures 50 thereby rotatably connecting the flexible strap 48 to the retractor blade 40.

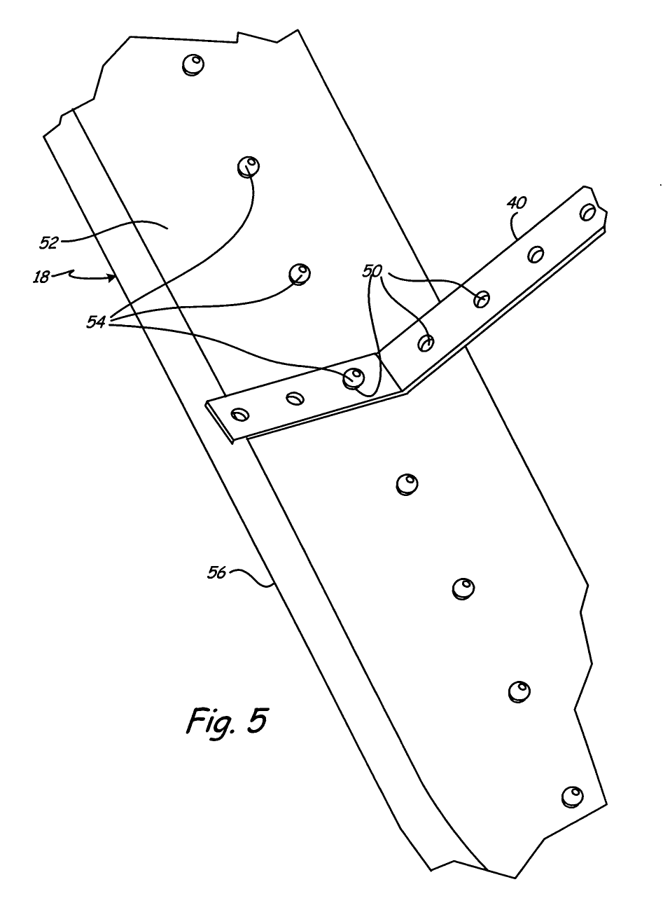

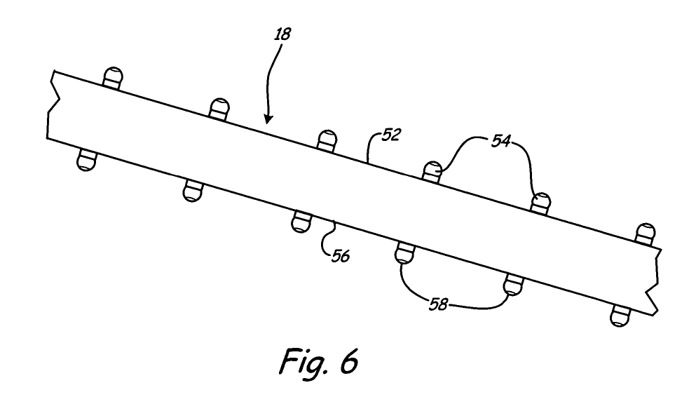

Referring to FIGS. 1-6, with the flesh engaging end 42 of the retractor blade 40 positioned within the incision 30, manual force is applied to the retractor 34 and preferably the flexible strap 48, which causes the retractor blade 40 to engage and retract the flesh from the surgical site to a selected position. With the flesh and the retractor blade 40 in the selected position, manual force is placed upon the flexible strap 40 to force one of the plurality of apertures 50 to accept one of a plurality of uniform pegs 54 extending from generally upper flat surface 52 of the support arm 18. In so doing, the flexible strap is placed in tension. By tension is meant that the strap is stretched until it is taut. The strap need not be elongated, although depending on the material some elongation will occur, and thereby place the strap in tension. The peg 68 and the plurality of uniform pegs 54 have a generally spherical end 47, 55 that has a diameter that is larger than a diameter of the plurality of apertures 50. Manual force is required to position the flexible stray 48 about either peg 46, 54 by expanding the diameter of the aperture 50 to accept the spherical ends 47, 55 of the pegs 46, 54.

The support arm 18 has a generally rectangular cross-section and includes a lower flat surface 56 that includes a plurality of uniform pegs 58 extending therefrom. The flexible strap 48 alternatively could be attached to one of the pegs 58 extending from the lower flat surface 56 to retain the skin, flesh and the retractor blade 40 in the selected retracting position.

Referring to FIGS. 1 and 2, the support arm 20 is generally a mirror image of the support arm 18 and has a generally rectangular cross section with an upper flat surface 60 and a lower flat surface 64. Both the upper flat surface 60 and the lower flat surface 64 include a plurality of uniform pegs 66, 68, respectively, extending therefrom. The uniform pegs 66, 68 can be forced into one of the plurality of apertures 50 in the flexible strap 48 to retain the retractor 34 to the support arm 20. The plurality of uniform pegs 58, 66, 68 have substantially the same configuration as the plurality of uniform pegs 54.

The flexible strap 48 is typically made of a polymeric material. The plurality of apertures 50 are positioned along the length of the flexible strap 48. However, the flexible member 48 may be of any construction such as woven, braided, non-woven material or flexible metal that allows the retractor blade 40 to move both laterally and vertically within the surgical site.

It should be understood that although a flexible strap 48 with a plurality of apertures 50 for engaging the pegs is illustrated, other types of fastening devices that attach the flexible connectors to the support arms may be used in the method of the present invention including but not limited to snaps, a hook and loop fastening system such as Velcro®, or a tie or clip on the flexible member. What is important is that the retractor starting from its attachment to either support arm 18 or 20 to the skin and flesh layers should not be rigid.

The procedure of the present invention permits the retractor blade to be moved as the patient's body is moved without necessitating repositioning the retractor blade, repositioning the attachment of the retractor to the retractor support or moving (adjusting) the retractor support. The flexible connector also needs to have sufficient integrity and strength to retain the retractor blade in a flesh retracted position. Although the flexible connector as shown extends from the retractor blade 40 to the support arm 18, the flexible connector does nbt necessarily have to extend from the blade 40 to the support arm 18. For example, only a portion of the flexible connector could be flexible while the remainder could be rigid as long as sufficient flexibility exists between the retractor blade 40 and the support arm 18 or 20 to be able to reposition the tibia 28 in relation to the femur 26. The flexible connector 48 may also be elastic or be made of resilient material as long as the connector is flexible. By flexible is meant that the surgeon may adjust the position of the knee-joint during surgery without having to reposition the retractor blade, reattach the retractor to the retractor support or adjust the position of the retractor support.

With the knee-joint exposed by retracting skin and flesh layers with retractors 34, 35, 36 and 37 supported and retained in the retracting position by the table mounted surgical support 12, one of a number of surgical procedures can be performed to the knee-joint 24 including a total knee-joint replacement surgery and a partial knee-joint replacement surgery. During the surgical procedure, the patella 32 is either removed or moved aside to gain access to the ends of the femur 28 and the tibia 26. Due to the flexible strap 48 of the retractors 34, 35, 36 and 37, the knee-joint can be repositioned without having to adjust the position of the support arm 18 or 20 or readjusting the retractor blade or reattaching the retractor to either support arm or both, 18 and 20.

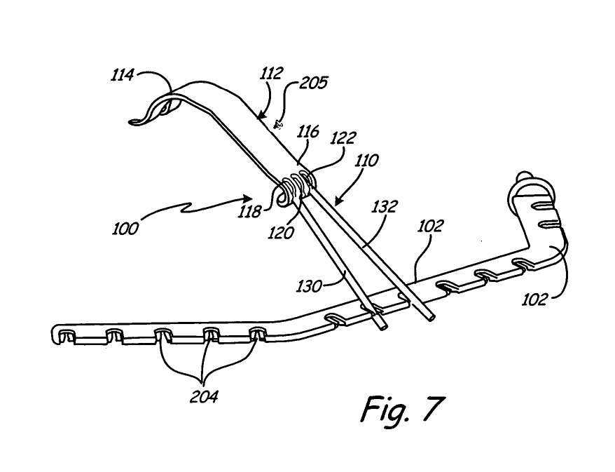

An alternative embodiment of the apparatus used in the method of surgical retraction of the present invention with a flexible connector is illustrated in FIG. 7 at 100. The apparatus 100 includes the same retractor support apparatus and clamping mechanism as described in the embodiment 10 and therefore will not be described in detail. A support arm 102 has the same general accurate configuration and rectangular cross section as the support arms 18 with the exception that a mechanism used to secure a flexible member to the support arm is different from the plurality of pegs 54, 58. The support arm 102 as illustrated in FIG. 7, would extend over the surgical table 13 along with another support arm (not shown), that is a mirror image of the support arm 102.

A retractor 110 includes a retractor blade 112 having a flesh engaging end 114 that is placed into the incision at the surgical site (not shown). A left flexible member 130 is positioned within a left slot 118 proximate a flexible member engaging end 116 of the retractor blade 112. The retractor blade 112 also includes a middle slot 120 and a right slot 122 wherein the left slot 118, the middle slot 120 and the right slot 122 all have substantially similar configurations. A right flexible member 132 is positioned within the right slot 122 wherein the left and right flexible members 130, 132 have substantially similar configurations. One skilled in the art will recognize that one longer flexible member looped through any two of the three slots would perform the same function as the separate left and right flexible members 130, 132, respectively.

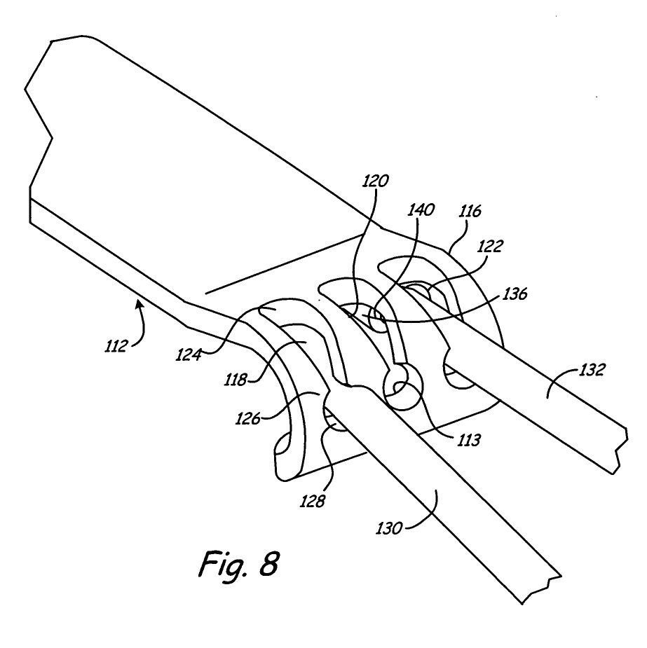

Referring to FIG. 8, each of the slots 118, 120, 122 in the retractor blade 112 have a top end 124 that accept wide segments one of which is illustrated at 136 of the flexible member 132. The flexible member 130 is of similar construction as the flexible member 132, and also includes a wide segment (not shown).

The width of the slots 118, 120, 122 taper down to a throat 126 that constricts an entrance to a retaining end 128. A shoulder, one of which is illustrated at 140, defined by the different diameters of the wide segment 136 and the remaining portion of the flexible member 132, engages a back surface 113 of the retractor blade 112. The diameter of the wide segments (not shown) 132 is greater than a diameter of the retaining end 128 which causes the shoulder 140 to engage a back surface 113 of the retractor blade 112 and retain the flexible members 130, 132 to the retractor blade 112.

The flexible members 130, 132 are typically made of a polymeric material in the form of a cord with a generally circular cross-section. However, the flexible members 130, 132 may be of any construction such as woven, braided, non-woven material, flexible metal or an elastic material and may have other cross-sectional configurations.

With the left and right flexible members 130, 132 retained within the left and right slots 118, 122, respectively, the flesh engaging end 114 of the retractor blade 112 is positioned within the incision (not shown). Manual force is applied to the retractor 112 and preferably with equal force upon the left and right flexible members 130, 132 which causes the retractor blade 112 to engage and retract the skin and flesh from the surgical site to a selected position.

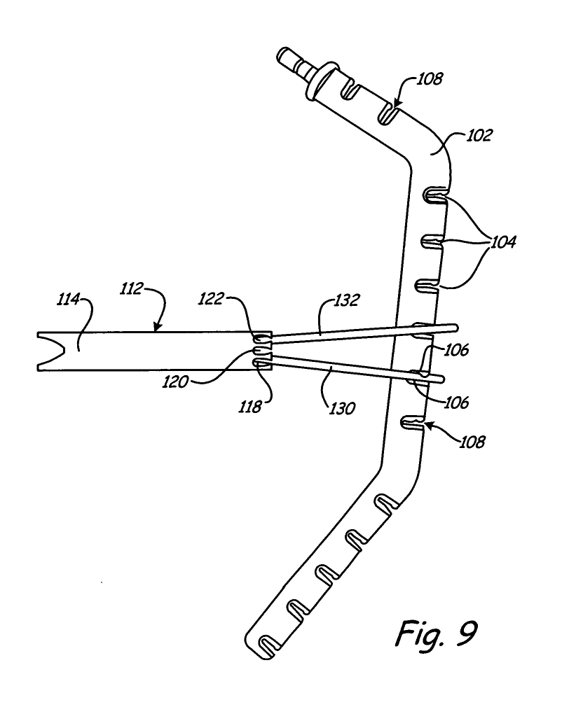

Referring to FIG. 9, with the skin, flesh and the retractor blade 112 in the selected position, manual downward force is placed upon the left and right flexible members 130, 132 to force the left and right flexible members 130, 132, each into one of a plurality of generally V-shaped notches 104 in the support arm 102 and into a frictional engagement with side surfaces 106 defining the V-shaped notches.

The generally V-shaped notches 104 preferably include a constricted entrance 108 which requires manual force to position the flexible members 130, 132 into the notches 104. The constricted entrance 108 also retains the retractor blade 112 to the support arm 102 in the event that the retractor blade 112 accidentally disengages from the flesh while the flexible members 130 are under tension. The generally V-shaped notches 104 expand past the constricted entrance 108 to allow the flexible member 130, 132 to be slidably positioned therein. A gap between the side surfaces 106 of the generally V-shaped notches 104 gradually reduce to a distance that results in a frictional engagement or pinching of the flexible members 130, 132 and retains the flexible members 130, 132 within the generally V-shaped notch 104.

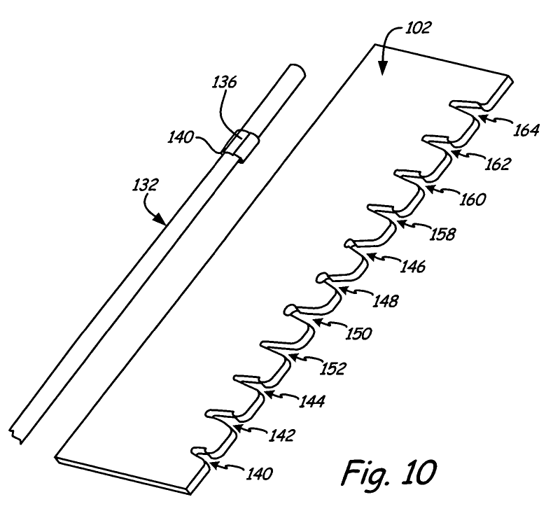

Referring to FIG. 10, the generally V-shaped notches 104 alternately may have other different configurations that frictionally retain the flexible members 130, 132 therein. The generally V-shaped notches can be shallow, medium length or elongated with a severely constricted entrance as illustrated at 140, 142 and 144, respectively. The generally V-shaped notches can be shallow, medium length or elongated notches 146, 148, 150, 152, all of which have non-constricted entrances that gradually taper to a generally circular cross-sectional end that frictionally engages the flexible member or may taper to a point. Finally, the generally V-shaped notches can have an entrance that has varying constrictions as illustrated at 158, 160 and 162, respectively.

The secured and spaced apart left and right flexible members 130, 132 restrict lateral movement of the retractor blade 112 within the surgical site because one flexible member 130, 132 will always be in tension depending upon the movement of the retractor blade 112. While restricting lateral movement, the retractor blade 112 is allowed to vertically move because of the flexible characteristics of the left and right flexible members 130, 132.

One skilled in the art will also recognize that only one flexible member secured to both the retractor blade 112 and the support arm 102 is required to practice the present invention. The single flexible member can be positioned within the left, middle or right slot 118, 120 and 122, respectively, in the retractor blade 112 while being capable of retracting skin and flesh.

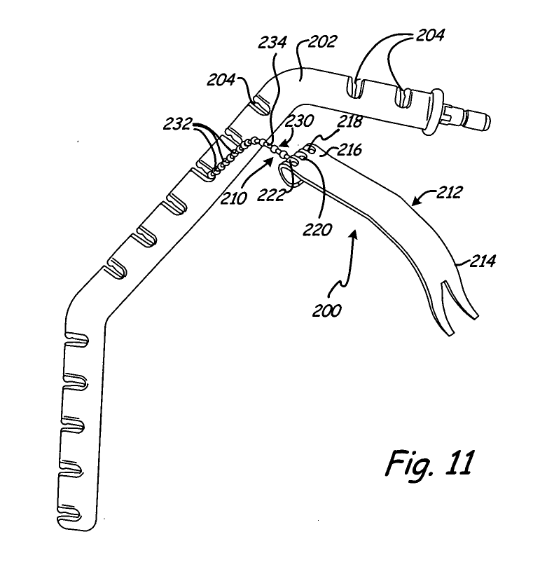

Another alternative embodiment of the apparatus used in the method of surgical retraction of the present with a flexible connector is illustrated in FIG. 11 at 200. The apparatus 200 includes the same retractor support apparatus and clamping mechanism as described in the embodiment 10 and therefore will not be described in detail. A support arm 202 has the same general accurate configuration, having a rectangular cross section with a plurality of notches 204 as the support arms 102. The support arm 202 as illustrated in FIG. 11, would extend over the surgical table 13 along with another support arm (not shown), that is a mirror image of the support arm 202.

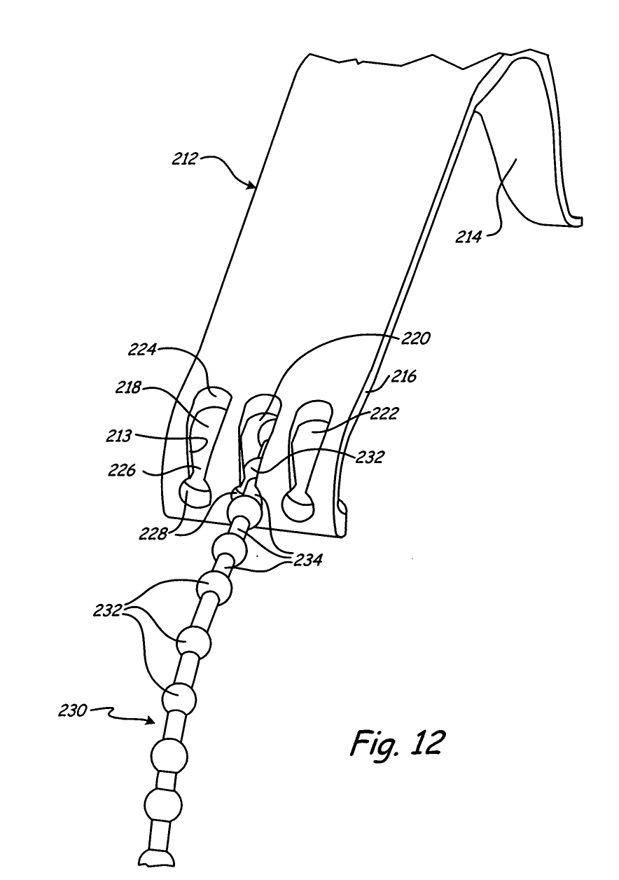

A retractor 210 includes a retractor blade 212 having a flesh engaging end 214 that is positioned into the incision at the surgical site (not shown). A beaded chain 230 having a plurality of beads 232 connected by narrow linkages 234 is positioned and retained within a middle slot 220 proximate a flexible member engaging end 216 of the retractor blade 212. The retractor blade 212 also includes a left slot 218 and a right slot 222 where the left slot 218, the middle slot 220 and the right slot 222 all have substantially similar configurations. Referring to FIG. 12, each of the slots 218, 220, 222 in the retractor blade 212 have a top end 224 that pass the beads 232 of the beaded chain 230 therethrough. The width of the slots 218, 220, 222 taper down to a throat 226 that constricts an entrance to a retaining end 228 that allows the linkages 234 to slide therein while engaging the beads 232 and thereby retaining the beaded chain 230 to the retractor blade 212. A diameter of the beads 234 is greater than a diameter of the retaining end 228 which causes the beads 234 to engage a back surface 213 of the retractor blade 212 and retain the beaded chain 230 to the retractor blade 212.

With the beaded chain 230 retained within the middle slot 220 the flesh engaging end 214 of the retractor blade 212 is positioned within the incision (not shown). Manual force is applied to the retractor 212 and preferably upon the beaded chain 230, which causes the retractor blade 212 to engage and retract the skin and flesh from the surgical site to a selected position.

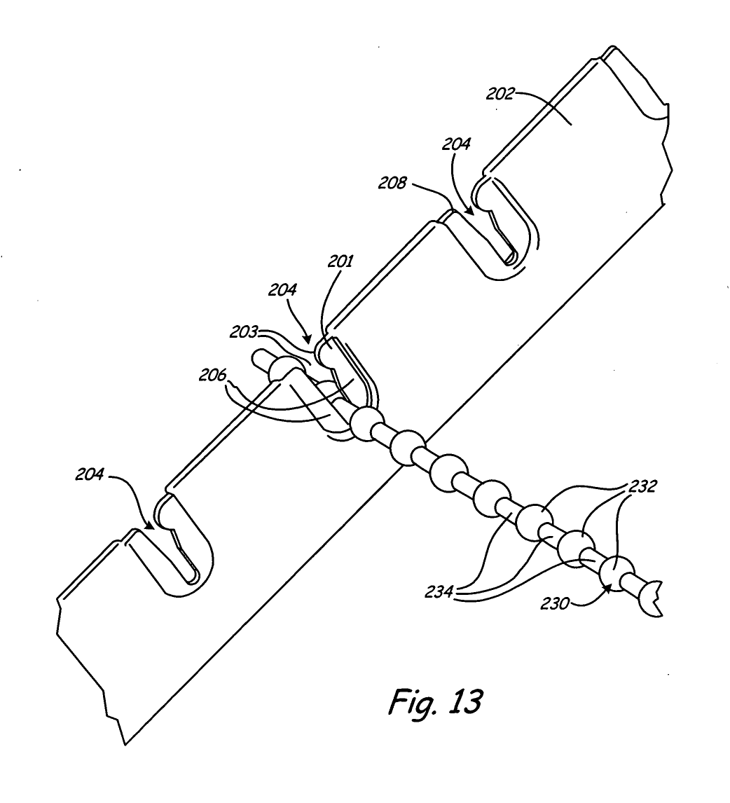

Referring to FIG. 13, with the skin, flesh and the retractor blade 212 in the selected position, manual downward force is placed upon the beaded chain 230 to force the beaded chain 230 into one of a plurality of generally V-shaped notches 204 in the support arm 202 and into an engagement with upper and lower surfaces 201, 203 of the support arm 202 wherein a gap between side surfaces 206 is less than the diameters of the beads 232 which results in an engagement between the surfaces 201, 203 and the beads 232 and thereby retains the retractor blade 212 in the selected position.

The generally V-shaped notches 204 preferably include a constricted entrance 208 which allows the linkage 234 to pass into the slots 204. The generally V-shaped slots 204 expand past the constricted entrance 208 to allow the beaded chain 230 to be slidably positioned therein. A gap between the side surfaces 206 of the generally V-shaped slots 204 gradually reduce to gaps such that the upper and lower surfaces 201, 203 engage the beads 232 and retain the beaded chain 230 within the generally V-shaped slots 204 and the retractor blade 212 in the selected retracting position.

It is also within the scope of the present invention to position two or more beaded chains 230 within the slots 218, 220, 222 to minimize lateral movement of the retractor blade 212 within the incision. The flexible beaded chains 230 allow for vertical positioning of the retractor blade 212 within the surgical site without having to reposition the beaded chains 230 on the retractor support arm 202.

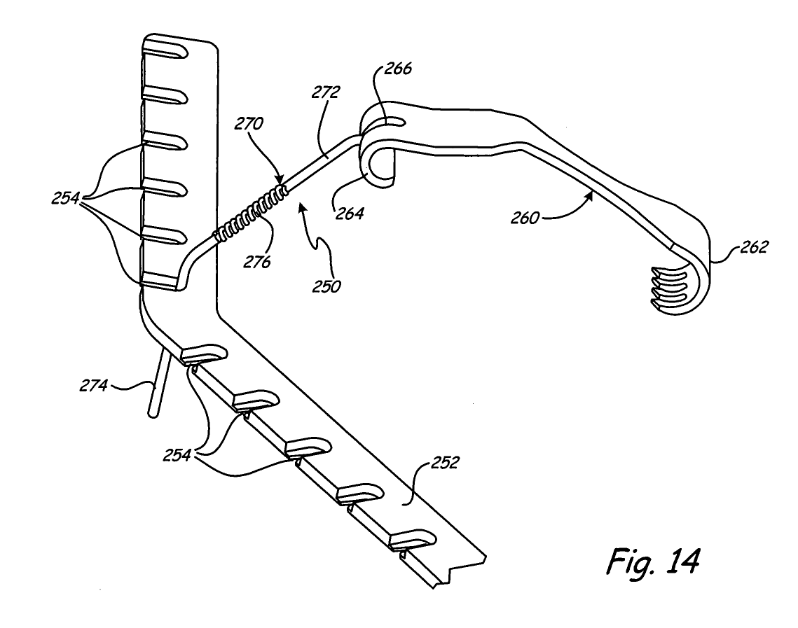

Another alternative embodiment of the apparatus used in the method of surgical retraction of the present with a flexible connector is illustrated in FIG. 14 at 250. The apparatus 250 includes the same retractor support apparatus and clamping mechanism as described in the embodiments 10 and therefore will not be described in detail. A support arm 252 has the same general accurate configuration, having a rectangular cross section with a plurality of notches 254 as the support arms 102. The support arm 252 as illustrated in FIG. 14, would extend over the surgical table 13 along with another support arm (not shown), that is a mirror image of the support arm 252.

A retractor 260 includes a retractor blade 262 having a flesh engaging end 214 that is positioned into the incision at the surgical site (not shown). A front portion 272 of a connecting member 270 is retained within a slot 266 of a retaining end 264 of the retractor blade 262. The front portion 272 has an enlarged portion (not shown) that engages an inner surface 268 of the retractor blade 262 proximate the slot 266 and retains the connecting member 270 to the retractor blade 262. A back portion 274 of the connecting member 270 is retained within the notches 254 as previously described with a frictional engagement.

The connecting member 270 also includes a spring 276 that connects the front portion 272 to the back portion 274. The front and back portion 272, 274 can be constructed of the flexible polymeric material as previously described where the spring provides additional flexibility and the capability of further elongating the connecting member 270.

The front and rear portions 272, 274 can also be constructed of a rigid or non-stretchable material such as a cable where the cable can be constructed of stainless steel. Where the front and rear portions 272, 274 are not stretchable and/or rigid, the spring provides the flexibility to adjust the position of the surgical site without having to reposition the retractor 262 on the support 252.

Although the present invention has been described with reference to preferred embodiments, workers skilled in the art will recognize that changes may be made in form and detail without departing from the spirit and scope of the invention.

1. A method of retracting flesh from a surgical site, the method comprising:
mounting a retractor support to the surgical table;
positioning the retractor support proximate the surgical site;
incising flesh layers proximate the surgical site;
retracting flesh layers by applying a retractor comprising a retractor blade having a flexible member attached thereto against the flesh layers; and
attaching the flexible member in tension to the retractor support to retain the retractor and flesh thereby exposing the surgical site.

2. The method of claim 1 and mounting the retractor support to a rail of the surgical table.

3. The method of claim 1 wherein the flexible member includes a plurality of apertures along a length thereof and wherein the retractor support comprises a plurality of pegs along a length thereof and attaching the flexible member in tension by inserting one of the pegs in a selected aperture to retain the retractor.

4. The method of claim 1 wherein the flexible member comprises a compressible cord and manually positioning the compressible cord within a generally V-shaped notch in the retractor support until the compressible cord engages side surfaces of the V-shaped notch, thereby retaining the retractor against the flesh.

5. The method of claim 1 and wherein the retractor comprises a second flexible member in tension attached to the retractor blade and wherein both flexible members are secured to the retractor support to retain the retractor against the flesh.

6. The method of claim 5 wherein the flexible members are compressible and manually positioning the flexible members within generally V-shaped notches that are spaced apart on the retractor support such that the flexible members engage side surfaces of the spaced apart V-shaped notches, thereby retaining the retractor blade against the flesh.

7. The method of claim 6 wherein the compressible cords are removably attached to the retractor blade.

8. The method of claim 1 and wherein the flexible member comprises a beaded chain and positioning the beaded chain in tension within one of a plurality of generally V-shaped notches in the retractor support to retain the retractor against the flesh.

9. The method of claim 8 and wherein the beaded chain is removably attached to the retractor blade.

10. The method of claim 1 and wherein the flexible member comprises a spring.

11. The method of claim 1 and wherein the surgical site comprises a knee-joint.

12. An apparatus for retracting flesh from a surgical site on a patient supported by a surgical table, the apparatus comprising:
a retractor support mounted to the surgical table, the retractor support including a plurality of pegs extending therefrom;
a retractor blade; and
a flexible member attached to the retractor blade and having a plurality of apertures therein such that the retractor blade is manually positioned against the flesh and the flexible member is placed in tension and one of the pegs is inserted into one of the apertures.

13. The apparatus of claim 12 and wherein the retractor blade comprises:
a flesh engaging end; and
a flexible member engaging end and wherein the flexible member is removably attached to the retractor blade proximate the flexible member engaging end.

14. The apparatus of claim 13 and wherein the retractor blade includes a peg proximate the flexible member engaging end extending into one of the plurality of apertures.

15. The apparatus of claim 12 and wherein the retractor support comprises:
a generally vertical post mounted to the surgical table; and
a retractor support apparatus mounted to the generally vertical post and wherein the retractor support apparatus comprises a clamp engaging left and right support arms extending therefrom and wherein when the clamp is in a non-clamping position the left and right support arms are movable independent of each other and wherein when the clamp is in the clamping position the left and right support arms are secured in selected positions.

16. An apparatus for retracting skin and flesh from a surgical site on a patient supported by a surgical table, the apparatus comprising:
a retractor support mounted to the surgical table and wherein the retract support comprises a plurality of notches therein; and
a retractor blade; and
at least one flexible member attached to the retractor blade such that the retractor blade is manually positionable against the flesh and the flexible member is placed in tension and within one of the notches thereby frictionally securing the flexible member to the retractor support.

17. The apparatus of claim 16 and wherein the retractor blade comprises:
a flesh engaging end; and
a flexible member engaging end and wherein the flexible member is removably attached to the retractor blade proximate the flesh engaging end.

18. The apparatus of claim 17 and wherein the retractor blade further comprises a plurality of slots proximate the flexible member engaging end.

19. The apparatus of claim 18 and further comprising two flexible members and wherein each flexible member is separately secured in tension within one of the plurality of slots in the retractor blade at one end and within the spaced apart notches in the retractor support at another end.

20. The apparatus of claim 16 and wherein the plurality of notches in the retractor support comprise surfaces that are spaced apart and gradually converge to a point such that as the flexible member is manually forced into one of the notches the slot the surfaces frictionally engage and retain the flexible member therein.

21. An apparatus for retracting skin and flesh from a surgical site on a patient supported by a surgical table having a side rail, the apparatus comprising:
a retractor support mounted to the side rail of the surgical table, the retractor support including a plurality of notches therein;
a retractor blade; and
a beaded chain attached to the retractor blade such that the retractor blade is manually positionable against the flesh and the beaded chain is placed in tension within one of the notches with the diameter of the beads being greater than a width of a proximal end of the slot thereby securing the beaded chain to the retractor support.

22. The apparatus of claim 21 and wherein the retractor blade comprises:
a flesh engaging end; and
a chain engaging end and wherein the beaded chain is removably attached to the retractor blade proximate the chain engaging end.

23. The apparatus of claim 22 and wherein the retractor blade further includes a plurality of slots and wherein the slots include a wide end and a narrow end positioned within the chain engaging end and wherein the beaded chain is disposed through one of the slots and is secured in tension by engaging the beads with the narrow end of the slot.
